(19) United States
(12) Patent Application Publication
(10) Pub. No.: US 2026/0263445 A1
(43) Pub. Date: Sep. 10, 2026

(54) TGF-BETA INHIBITOR FOR USE IN THE TREATMENT OF DEDIFFERENTIATED LIPOSARCOMA

(71) Applicants: Institut Curie, Paris (FR); Institut National de la Sante et de la recherche Medicale, Paris (FR)

(72) Inventors: Sarah WATSON, Paris (FR); Nadège GRUEL, Paris (FR); Chloé QUIGNOT, Paris (FR)

(21) Appl. No.: 19/167,174

(22) PCT Filed: Mar. 20, 2024
(86) PCT No.: PCT/IB2024/000154

§ 371 Date: Sep. 19, 2025

(30) Foreign Application Priority Data
Mar. 21, 2023 (WO) PCT/IB2023/052779

(51) Int. Cl. A61K 31/4709; A61K 31/4439; A61K 45/06; A61P 35/00

(52) U.S. Cl. CPC A61K 31/4709; A61K 31/4439; A61K 45/06; A61P 35/00

(57) ABSTRACT

The inventors investigated new mechanism of adipocytic differentiation in dedifferentiated liposarcomas (DDLPS). The inventors demonstrate that DDLPS tumor cells are vulnerable to TGFP inhibition and provide rationale for the development of therapeutic strategies based on TGFP inhibition. Treatment of DD tumor cells with TGFP impairs adipocytic differentiation of DD cells, whereas TGFP inhibitors restore their adipocytic phenotype in vitro. The treatment of mice engrafted with DDLPS patient-derived xenografts (PDX) with inhibitor of TGFP reduce tumor growth and restore adipocytic differentiation of DD cells. Thus, the present invention more particularly relates to TGFP inhibitor for use in the treatment of DDLPS, particularly of advanced DDLPS.

### FIELD OF THE INVENTION

The invention relates to methods and pharmaceutical compositions for the treatment of dedifferentiated liposarcomas (DDLPS), particularly advanced dedifferentiated liposarcomas.

### BACKGROUND OF THE INVENTION

Dedifferentiated liposarcomas (DDLPS) are the most frequent high-grade soft tissue sarcoma in adults. From a pathological point of view, these tumors are composed of high-grade undifferentiated tumor cells (DD), often showing an abrupt transition from a compartment of well-differentiated adipocytic tumor cells (WD). Tumor cells from both WD and DD compartments are characterized by recurrent MDM2 amplification, but their cellular origin and the molecular mechanisms associated with dedifferentiation are poorly understood.

The inventors performed an integrated molecular analysis of tumors collected from 11 patients undergoing surgery for primary untreated DDLPS. DDLPS tumors were analyzed by single-cell RNA sequencing (scRNAseq) and bulk RNA sequencing on paired WD and DD samples from the same tumors. Results were validated in vitro and in vivo in an additional cohort of human tumors, patient-derived xenografts and DDLPS cell lines.

Through RNA-sequencing of 102,753 individual cells from 11 primary DDLPS lesions, major cell clusters were identified based on unsupervised clustering of gene expression profiles and canonical markers. They include 31 tumor microenvironment clusters and 11 tumor cell clusters. A cluster of tumor cells from the WD compartment is characterized by signatures of early adipocytic progenitors, previously identified as TGFβ-dependent, DPP4-positive stromal progenitors. The inventors show that these cells harbor specifically the truncal genomic alterations of the cancer, with further subclonal mutations identifiable in both WD and DD compartments of DDLPS. Furthermore, these cells have multipotent properties and their differentiation towards the adipocytic lineage is inhibited by TGFβ. Treatment of DD tumor cells with TGFβ inhibitors restores their adipocytic phenotype in vitro and in vivo. The inventors provide the first single-cell atlas of human DDLPS tumor and microenvironment and identify a population of adipocytic tumor progenitors at the origin of both WD and DD compartments.

The inventors investigated new mechanism of adipocytic differentiation in DDLPS to find curative options for DDLPS and advanced DDLPS for which no therapeutic solutions are available. The inventors demonstrate that DDLPS tumor cells are vulnerable to TGFβ inhibition and provide rationale for the development of therapeutic strategies based on TGFβ inhibition in advanced DDLPS. Ex vivo adipocytic differentiation of DD tumor cells from 2 DDLPS PDX models is prevented in the presence of TGFβ both at the transcriptomic and phenotypic level, whereas treatment with TGFβ inhibitors such as galunisertib or SB43152 restores adipocytic differentiation. Treatment of DD tumor cells with TGFβ impairs adipocytic differentiation of DD cells, whereas TGFβ inhibitors restore their adipocytic phenotype in vitro.

The treatment of DDLPS patient-derived xenografts (PDX) with inhibitors of TGFβ has been found to reduce tumor growth and restore adipocytic differentiation of DD cells. 7 nude mice engrafted with the DDLPS PDX Sil10AS are treated with TGFβ inhibitor galunisertib. The tumor growth rate is compared to a control group of 7 nude mice engrafted with the same PDX model and treated with a vehicle. At the end of the experiment, tumor differentiation are assessed at the microscopic and transcriptomic level.

There is no disclosure in the art of the inhibition of differentiation towards the adipocytic lineage by TGFβ in DDLPS; there is even less disclosure about the restoration of adipocytic differentiation in DDLPS in DD tumors cells and the reduction of DDLPS tumor growth rate upon expo inhibition of TGFβ. Accordingly, there is no disclosure either of TGFβ inhibitors for use in the treatment of DDLPS and advanced DDLPS.

### SUMMARY OF THE INVENTION

The inventors demonstrate for the first time the role of TGFβ pathway in DDLPS. They show further that inhibition of TGFβ pathway restores adipocytic differentiation in DDLPS tumors and that DDLPS tumor cells are vulnerable to TGFβ inhibition.

Accordingly, the invention relates to methods and pharmaceutical compositions for the treatment of dedifferentiated liposarcomas (DDLPS), particularly advanced dedifferentiated liposarcomas. In particular, the invention is defined by the claims.

In a first aspect, the invention relates to a TGFβ inhibitor for use in the treatment of a dedifferentiated liposarcoma (DDLPS) in a subject in need thereof. Indeed, as explained above and show in the experimental part, inhibition of TGFβ pathway results in a reduced tumor growth rate.

In particular independent features, said TGFβ inhibitor for use in the treatment of a dedifferentiated liposarcoma (DDLPS) can be:
selected from: antisense oligonucleotides preventing TGF-β synthesis, ligand traps and neutralizing antibodies preventing a TGF-β ligand from binding to the receptors; small molecule TGF-β inhibitors that inhibit receptor kinase activity and/or prevent TGFβ receptor mediated signal transduction; all these kinds of TGFβ inhibitors being known as effective in inhibiting TGFβ pathway and developed, used or under development in other therapeutic area.
selected from galunisertib, SB431542, 2G7, 1D11, LY2109761, IN-1130, LY364947, PF-03446962, Ki 26894, SD208, SB505124, LY550410, AP12009, AP1104, AP15012, ISTH0036, fresolimumab, LY2382770, BX-BE0057, luspatercept, bintrafusp alfa, TQB2858, CAT-192, P144, belagenpumatucel-L or gemogenovatucel-T, or a combination thereof. These compounds are well known TGFβ inhibitors as defined below.
for use in the treatment of an advanced dedifferentiated liposarcoma.

Also, stemming for the unexpected discovery of the role of TGFβ in DDLPS, in a second aspect, the invention relates also to pharmaceutical composition comprising at least one TGFβ inhibitor and at least one pharmaceutical acceptable carrier for use in the treatment of a dedifferentiated liposarcoma in a subject in need thereof.

In particular independent features, said pharmaceutical composition:
can comprise at least one TGFβ inhibitor selected from galunisertib, SB431542, 2G7, 1D11, LY2109761, IN-1130, LY364947, PF-03446962, Ki 26894, SD208, SB505124, LY550410, AP12009, AP1104, AP15012, ISTH0036, fresolimumab, LY2382770, BX-BE0057, luspatercept, bintrafusp alfa, TQB2858, CAT-192, P144, belagenpumatucel-L or gemogenovatucel-T, or a combination thereof.
is for use in treating an advanced dedifferentiated liposarcoma.

According to a third aspect, the invention relates to a method of treating a dedifferentiated liposarcoma in a subject in need thereof, comprising administering to the subject a therapeutically effective amount of at least one TGFβ inhibitor, which can comprise the following independent particular features:
the at least one TGFβ inhibitor is selected from galunisertib, SB431542, 2G7, 1D11, LY2109761, IN-1130, LY364947, PF-03446962, Ki 26894, SD208, SB505124, LY550410, AP12009, AP1104, AP15012, ISTH0036, fresolimumab, LY2382770, BX-BE0057, luspatercept, bintrafusp alfa, TQB2858, CAT-192, P144, belagenpumatucel-L or gemogenovatucel-T, or a combination thereof.

### DETAILED DESCRIPTION OF THE INVENTION

The inventors investigated new mechanism of adipocytic differentiation in DDLPS to find curative options for DDLPS and advanced DDLPS. The inventors demonstrate for the first time that DDLPS tumor cells are vulnerable to TGFβ inhibition and, consistent with this discovery, provide rationale for the development of therapeutic strategies based on TGFβ inhibition in advanced DDLPS. Ex vivo adipocytic differentiation of DD tumor cells from 2 DDLPS PDX models is prevented in the presence of TGFβ both at the transcriptomic and phenotypic level, whereas treatment with TGFβ inhibitors is found to restore adipocytic differentiation. Treatment of DD tumor cells with TGFβ impairs adipocytic differentiation of DD cells, whereas TGFβ inhibitors restores their adipocytic phenotype in vitro. Even more surprisingly, the treatment of mice engrafted with DDLPS patient-derived xenografts (PDX) with a inhibitor of TGFβ is found to reduce tumor growth and restore adipocytic differentiation of DD cells. Altogether, the present invention highlights the role of TGFβ in adipocytic differentiation in DDLPS, particularly advanced DDLPS and the use of TGFβ inhibitors in the treatment of DDLPS, particularly advanced DDLPS.

Accordingly, the invention relates to the targeting of TGFβ in the treatment of dedifferentiated liposarcomas (DDLPS), particularly advanced dedifferentiated liposarcomas (DDLPS).

### Compounds for Use in the Treating DDLPS

Accordingly, in a first aspect, the invention relates to a TGFβ inhibitor for use in the treatment of a dedifferentiated liposarcoma (DDLPS) in a subject in need thereof.

In some embodiment, the invention relates to TGFβ inhibitor for use in the treatment of advanced dedifferentiated liposarcomas (DDLPS) in a subject in need thereof.

As used herein, the terms “subject”, “individual” or “patient” are interchangeable and refer to a mammal. Typically, a subject according to the invention refers to any subject, preferably human. In a particular embodiment, the term “subject” refers to a subject afflicted or at risk to be afflicted with cancer. In a particular embodiment, the term “subject” refers to a subject afflicted or at risk to be afflicted with a dedifferentiated liposarcoma (DDLPS). In a particular embodiment, the term “subject” refers to a subject afflicted or at risk to be afflicted with an advanced dedifferentiated liposarcomas (DDLPS).

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “dedifferentiated liposarcoma” or “DDLPS” has its general meaning in the art and refers to any dedifferentiated liposarcoma (DDLPS), the most frequent high-grade soft tissue sarcoma in adults. From a pathological point of view, these tumors are composed of high-grade undifferentiated tumor cells (DD), often showing an abrupt transition from a compartment of well-differentiated adipocytic tumor cells (WD) (Watson et al., 2022; Gruel et al., 2023). Well-differentiated and dedifferentiated liposarcomas are largely characterized by the presence of several massive abnormal chromosomes called neochromosomes, which carry dozens of copies of the oncogenes MDM2 and CDK4. The term “dedifferentiated liposarcoma” include advanced dedifferentiated liposarcoma.

The term “TGFβ” has its general meaning in the art and refers to a Transforming growth factor-beta (TGF-β), a multifunctional cytokine belonging to the transforming growth factor superfamily that includes three different mammalian isoforms (TGF-β 1, 2 and 3). TGFβ is a prototype for a large family of growth and differentiation factors that regulate development and involved in diverse physiological processes, including embryonic development, proliferation, differentiation, metastasis, apoptosis, immune responses, and many other important processes in malignancy. TGF-β family members activate transmembrane serine/threonine receptor kinases, thereby initiating a signaling cascade via Smads, a class of intracellular signaling effectors that regulate gene expression. TGF-β is a potent inducer of growth arrest in many cell types, including epithelial cells. This activity is the basis of the tumor suppressor role of the TGF-β signaling system in carcinomas. Other activities, including TGF-β-induced epithelial-to-mesenchymal differentiation, contribute to cancer progression.

As used herein, the term “TGFβ inhibitor” refers to any compound selected from the group consisting of but not limited to compounds targeting Transforming growth factor-beta (TGF-β), TGF-β 1, 2 and 3, TGF-β receptors, TGF-β ligands and TGF-β signaling cascade such as via Smads signaling system. The term “TGFβ inhibitor” refers to compounds that bind to TGFβ, TGF-β receptor, TGF-β ligands or TGF-β signaling factors and function as potent antagonists of TGFβ. The term “TGFβ inhibitor” has its general meaning in the art and refers to a compound that selectively inactivates TGFβ. Typically, a TGFβ inhibitor is a small organic molecule, a polypeptide, an aptamer, an oligonucleotide (antisense oligonucleotides, siRNA, shRNA, DNA and RNA aptamers), or an antibody. TGFβ inhibitors are well-known in the art as such as described in Huang et al., 2021.

The term “TGFβ inhibitor” also refers to TGF-β pathway inhibitor such as inhibitors at ligand level: Antisense oligonucleotides (ASOs) delivered directly intravenously or engineered into immune cells to prevent TGF-synthesis such as AP12009, AP1104, AP15012, Lucanix™, and ISTH0036; ligand traps and neutralizing antibodies that prevent a TGF-β ligand from binding to the receptors such as GC1008, 2G7, 1D11, LY2382770, BX-BE0057, luspatercept, bintrafusp alfa (a bispecific antibody), TQB2858 (a bispecific antibody) and CAT-192 (memelimumab); vaccine-based strategy; small molecule TGF-β inhibitors that inhibit receptor kinase activity and prevent signal transduction such as SB431542, Ki 26894, SD-208, LY2109761, IN-1130, LY2157299, TEW-7197, LY364947 and PF-03446962; and intracellular peptide aptamers (and antagonists) and inhibitors described in Huang et al., 2021.

The term “TGFβ inhibitor” refers to any compound selected from but not limited to small molecule TGF-β inhibitors such as LY2157299 (galunisertib) targeting TBRI (ALK5), LY2109761 targeting TβRI/RII, LY364947 targeting TBRI/RII, SB431542 an inhibitor of the activin receptor-like kinase (ALK) receptors, TEW-7197 targeting TBRI (ALK5), SB505124, and LY550410, antisense oligonucleotides such as AP12009 (trabederson) and ISTH0036, antibody and ligand trap such as GC1008 (fresolimumab), LY2382770, BX-BE0057, luspatercept, bintrafusp alfa (a bispecific antibody targeting PD-L1 et TGFβ), TQB2858 (a bispecific antibody targeting PD-L1 et TGFβ) and P144 targeting TGFβ RI/II complex, vaccines target TGF-β pathway such as (formerly known as FANG™ vaccine) 10 belagenpumatucel-L (Lucanix™), Vigil™ (gemogenovatucel-T).

Cas Number, name, provider data or article citing some of the compounds useful in the context of the current invention are provided in table 1 below.

TABLE 1

| | CAS number/Name/reference |
|---|---|
| TGFβ inhibitor | (provider)/article citation |
| galunisertib | 700874-72-2 |
| SB431542 | 301836-41-9 |
| 2G7 | cited in Huang et al (2021) |
| 1D11 | # MA5-23795 (Thermofisher) |
| LY2109761 | 700874-71-1 |
| IN-1130 | 868612-83-3 |
| LY364947 | 396129-53-6 |
| PF-03446962 | 1463459-96-2 |
| (Ascrinvacumab) | |
| SB431542 | 301836-41-9 |
| Ki 26894 | 935761-30-1 |
| SD208 | 627536-09-8 |
| SB505124 | 694433-59-5 |
| LY550410 | cited in Huang et al (2021) |
| AP12009 | trabedersen |
| AP1104 | cited in Huang et al (2021) |
| AP15012 | cited in Huang et al (2021) |
| ISTH0036 | cited in Huang et al (2021) |
| fresolimumab | 948564-73-6 |
| LY2382770 | Afuco ™, cited in Huang |
| | et al (2021) |
| BX-BE0057 | #BE0057 (Bio X Cell) |
| luspatercept | 1373715-00-4 |
| bintrafusp alfa | 1918149-01-5 |
| TQB2858 | Xie L et al (2023) |
| CAT-192 | 272780-74-2 |
| P144 | 272105-42-7 |
| belagenpumatucel-L | 1151760-94-9 |
| gemogenovatucel-T | FANG ™ or VIGIL ™ cited |
| | in Huang et al (2021) |

Tests and assays for determining whether a compound is a TGFβ inhibitor are well known by the skilled person in the art such as described in Frey et al., 2022; Szymańska et al., 2020. Determining whether a compound is a TGFβ inhibitor may also be performed using by using recombinant TGFβ proteins, competitive binding assays and measuring the binding affinities, measuring activated TGF-β complexes with other factors to form a serine/threonine kinase complex that binds to TGF-β receptors. In a particular embodiment, a “TGFβ inhibitor” as used herein refers to a compound that is able to inhibit, or to significantly reduce, in cultured cells in vitro or ex vivo, signal transduction through the so called in the art canonical TGF-β pathway. In a more particular embodiment, a “TGFβ inhibitor” as used herein refers to a compound that is able to inhibit, or to significantly reduce, in cultured cells in vitro or ex vivo, phosphorylation level of SMAD2 and/or SMAD3 protein, in presence of an agonist of a TGF-β receptor (preferably TGF-β). In a more particular embodiment, said compound is found able to inhibit, or to significantly reduce, in cultured cells from dedifferentiated liposarcoma in vitro or ex vivo phosphorylation of SMAD2 and/or SMAD3 protein. In an even more particular embodiment, a TGFβ inhibitor useful in the treatment of a dedifferentiated liposarcoma (DDLPS) in a subject in need thereof is able to significantly reduce phosphorylation level of SMAD2 and/or SMAD3 protein of at least 5%, 10%, 20%, 20%, 30%, 40%, 50%, 60%, 70%, 80%, at least 90% or even 100% when compared to phosphorylation level observed in presence of an agonist TGF-β receptors (e.g. TGFβ) in the corresponding cultured cells, preferably cells from dedifferentiated liposarcoma. In another particular embodiment, a TGFβ inhibitor according to the invention is at least as effective as galunisertib and/or as SB431542 in reducing phosphorylation level of SMAD2 and/or SMAD3 protein. Antibodies and/or kits to detect and quantify said phosphorylation level are well known from the skilled in the art and are commercially available.

In another embodiment, the compound of the invention is an aptamer. Aptamers are a class of molecule that represents an alternative to antibodies in term of molecular recognition. Aptamers are oligonucleotide sequences with the capacity to recognize virtually any class of target molecules with high affinity and specificity. Such ligands may be isolated through Systematic Evolution of Ligands by Exponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996). Then after raising aptamers directed against the target of the invention as above described, the skilled man in the art can easily select those blocking or inactivating the target.

In another embodiment, the compound of the invention is an antibody (the term including “antibody portion”) directed against the target.

In one embodiment of the antibodies or portions thereof described herein, the antibody is a monoclonal antibody. In one embodiment of the antibodies or portions thereof described herein, the antibody is a polyclonal antibody. In one embodiment of the antibodies or portions thereof described herein, the antibody is a humanized antibody. In one embodiment of the antibodies or portions thereof described herein, the antibody is a chimeric antibody. In one embodiment of the antibodies or portions thereof described herein, the portion of the antibody comprises a light chain of the antibody. In one embodiment of the antibodies or portions thereof described herein, the portion of the antibody comprises a heavy chain of the antibody. In one embodiment of the antibodies or portions thereof described herein, the portion of the antibody comprises a Fab portion of the antibody. In one embodiment of the antibodies or portions thereof described herein, the portion of the antibody comprises a F(ab′) 2 portion of the antibody. In one embodiment of the antibodies or portions thereof described herein, the portion of the antibody comprises a Fc portion of the antibody. In one embodiment of the antibodies or portions thereof described herein, the portion of the antibody comprises a Fv portion of the antibody. In one embodiment of the antibodies or portions thereof described herein, the portion of the antibody comprises a variable domain of the antibody. In one embodiment of the antibodies or portions thereof described herein, the portion of the antibody comprises one or more CDR domains of the antibody.

As used herein, “antibody” includes both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. The antibody may be a human or nonhuman antibody. A nonhuman antibody may be humanized by recombinant methods to reduce its immunogenicity in man.

Antibodies are prepared according to conventional methodology. Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with antigenic forms of the target. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes.

Briefly, the antigen may be provided as synthetic peptides corresponding to antigenic regions of interest in the target. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′) 2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

This invention provides in certain embodiments compositions and methods that include humanized forms of antibodies. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria, which may be used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgGl, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Thus, as will be apparent to one of ordinary skill in the art, the present invention also provides for F(ab′) 2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′) 2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgG1, IgG2, IgG3 and IgG4. In a preferred embodiment, the compound of the invention is a Human IgG4.

In another embodiment, the antibody according to the invention is a single domain antibody. The term “single domain antibody” (sdAb) or “VHH” refers to the single heavy chain variable domain of antibodies of the type that can be found in Camelid mammals, which are naturally devoid of light chains. Such VHH are also called “Nanobody®”. According to the invention, sdAb can particularly be llama sdAb. The term “VHH” refers to the single heavy chain having 3 complementarity determining regions (CDRs): CDR1, CDR2 and CDR3. The term “complementarity determining region” or “CDR” refers to the hypervariable amino acid sequences which define the binding affinity and specificity of the VHH.

The VHH according to the invention can readily be prepared by an ordinarily skilled artisan using routine experimentation. The VHH variants and modified form thereof may be produced under any known technique in the art such as in-vitro maturation.

VHHs or sdAbs are usually generated by PCR cloning of the V-domain repertoire from blood, lymph node, or spleen cDNA obtained from immunized animals into a phage display vector, such as pHEN2. Antigen-specific VHHs are commonly selected by panning phage libraries on immobilized antigen, e.g., antigen coated onto the plastic surface of a test tube, biotinylated antigens immobilized on streptavidin beads, or membrane proteins expressed on the surface of cells. However, such VHHs often show lower affinities for their antigen than VHHs derived from animals that have received several immunizations. The high affinity of VHHs from immune libraries is attributed to the natural selection of variant VHHs during clonal expansion of B-cells in the lymphoid organs of immunized animals. The affinity of VHHs from non-immune libraries can often be improved by mimicking this strategy in vitro, i.e., by site directed mutagenesis of the CDR regions and further rounds of panning on immobilized antigen under conditions of increased stringency (higher temperature, high or low salt concentration, high or low pH, and low antigen concentrations). VHHs derived from camelid are readily expressed in and purified from the E. coli periplasm at much higher levels than the corresponding domains of conventional antibodies. VHHs generally display high solubility and stability and can also be readily produced in yeast, plant, and mammalian cells. For example, the “Hamers patents” describe methods and techniques for generating VHH against any desired target (see for example U.S. Pat. Nos. 5,800,988; 5,874,541 and 6,015,695). The “Hamers patents” more particularly describe production of VHHs in bacterial hosts such as E. coli (see for example U.S. Pat. No. 6,765,087) and in lower eukaryotic hosts such as moulds (for example Aspergillus or Trichoderma) or in yeast (for example Saccharomyces, Kluyveromyces, Hansenula or Pichia) (see for example U.S. Pat. No. 6,838,254).

In another aspect, the invention provides an antibody that competes for binding to the target with the antibody of the invention.

As used herein, the term “binding” in the context of the binding of an antibody to a predetermined antigen or epitope typically is a binding with an affinity corresponding to a KD of about 10-7 M or less, such as about 10-8 M or less, such as about 10-9 M or less, about 10-10 M or less, or about 10-11 M or even less when determined by for instance surface plasmon resonance (SPR) technology in a BIAcore 3000 instrument using a soluble form of the antigen as the ligand and the antibody as the analyte. BIACORE® (GE Healthcare, Piscaataway, NJ) is one of a variety of surface plasmon resonance assay formats that are routinely used to epitope bin panels of monoclonal antibodies. Typically, an antibody binds to the predetermined antigen with an affinity corresponding to a KD that is at least ten-fold lower, such as at least 100-fold lower, for instance at least 1,000-fold lower, such as at least 10,000-fold lower, for instance at least 100,000-fold lower than its KD for binding to a non-specific antigen (e.g., BSA, casein), which is not identical or closely related to the predetermined antigen. When the KD of the antibody is very low (that is, the antibody has a high affinity), then the KD with which it binds the antigen is typically at least 10,000-fold lower than its KD for a non-specific antigen. An antibody is said to essentially not bind an antigen or epitope if such binding is either not detectable (using, for example, plasmon resonance (SPR) technology in a BIAcore 3000 instrument using a soluble form of the antigen as the ligand and the antibody as the analyte), or is 100 fold, 500 fold, 1000 fold or more than 1000 fold less than the binding detected by that antibody and an antigen or epitope having a different chemical structure or amino acid sequence.

Additional antibodies can be identified based on their ability to cross-compete (e.g., to competitively inhibit the binding of, in a statistically significant manner) with other antibodies of the invention in standard antigen binding assays. The ability of a test antibody to inhibit the binding of antibodies of the present invention to the target demonstrates that the test antibody can compete with that antibody for binding to the target; such an antibody may, according to non-limiting theory, bind to the same or a related (e.g., a structurally similar or spatially proximal) epitope on the target as the antibody with which it competes. Thus, another aspect of the invention provides antibodies that bind to the same antigen as, and compete with, the antibodies disclosed herein. As used herein, an antibody “competes” for binding when the competing antibody inhibits the target binding of an antibody or antigen binding fragment of the invention by more than 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% in the presence of an equimolar concentration of competing antibody.

In other embodiments the antibodies or antigen binding fragments of the invention bind to one or more epitopes of the target. In some embodiments, the epitopes to which the present antibodies or antigen binding fragments bind are linear epitopes. In other embodiments, the epitopes to which the present antibodies or antigen binding fragments bind are non-linear, conformational epitopes.

In one embodiment, the TGFβ inhibitor of the invention is a TGFβ expression inhibitor.

The term “expression” when used in the context of expression of a gene or nucleic acid refers to the conversion of the information, contained in a gene, into a gene product. A gene product can be the direct transcriptional product of a gene (e.g., mRNA, tRNA, IRNA, antisense RNA, ribozyme, structural RNA or any other type of RNA) or a protein produced by translation of a mRNA. Gene products also include messenger RNAs, which are modified, by processes such as capping, polyadenylation, methylation, and editing, and proteins modified by, for example, methylation, acetylation, phosphorylation, ubiquitination, SUMOylation, ADP-ribosylation, myristilation, and glycosylation.

An “inhibitor of expression” refers to a natural or synthetic compound that has a biological effect to inhibit the expression of a gene. An “inhibitor of expression” refers to any compound that has a biological effect to inhibit the expression of a target gene and/or the expression of target protein. In one embodiment of the invention, said inhibitor of expression is a short hairpin RNA (shRNA), a small inhibitory RNA (siRNA), or an antisense oligonucleotide. Preferably, the inhibitor of expression is a siRNA or a shRNA.

The target expression inhibitors for use in the present invention may be based on antisense oligonucleotide constructs. Anti-sense oligonucleotides, including anti-sense RNA molecules and anti-sense DNA molecules, would act to directly block the translation of the target mRNA by binding thereto and thus preventing protein translation or increasing mRNA degradation, thus decreasing the level of the target proteins, and thus activity, in a cell. For example, antisense oligonucleotides of at least about 15 bases and complementary to unique regions of the mRNA transcript sequence encoding the target can be synthesized, e.g., by conventional phosphodiester techniques and administered by e.g., intravenous injection or infusion. Methods for using antisense techniques for specifically alleviating gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as a target expression inhibitors for use in the present invention. The target gene expression can be reduced by contacting the subject or cell with a small double stranded RNA (dsRNA), or a vector or construct causing the production of a small double stranded RNA, such that the target expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, MT. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Short hairpin RNA (shRNA) or Small inhibitory RNAs (siRNAs) can function as inhibitors of gene expression for use in the invention. Gene expression can be reduced with a small double stranded RNA (dsRNA), or a vector or construct causing the production of a small double stranded RNA, such that gene expression is specifically inhibited (i.e. RNA interference or RNAi). Methods for selecting an appropriate dsRNA or dsRNA-encoding vector are well known in the art for genes whose sequence is known.

Ribozymes can also function as target expression inhibitors for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of the target mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides (ODNs) and ribozymes useful as target inhibitors can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing the target. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in KRIEGLER (A Laboratory Manual,” W.H. Freeman C. O., New York, 1990) and in MURRY (“Methods in Molecular Biology,” vol. 7, Humana Press, Inc., Cliffton, N.J., 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g., SANBROOK et al., “Molecular Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a further aspect, the invention relates to a method of treating a dedifferentiated liposarcoma (DDLPS) in a subject in need thereof, comprising administering to the subject a therapeutically effective amount of a TGFβ inhibitor.

In some embodiments, the invention relates to a method of treating an advanced dedifferentiated liposarcoma (DDLPS) in a subject in need thereof, comprising administering to the subject a therapeutically effective amount of a TGFβ inhibitor.

In some embodiments, the TGFβ inhibitor and/or pharmaceutical composition according to the invention is used in combination with cancer therapies. In particular, the compound and/or pharmaceutical composition of the invention may be administered in combination with targeted therapy, immunotherapy such as immune checkpoint therapy and immune checkpoint inhibitor, co-stimulatory antibodies, chemotherapy and/or radiotherapy.

As used herein, the term “immunotherapy” refers to a cancer therapeutic treatment using the immune system to reject cancer. The therapeutic treatment stimulates the patient's immune system to attack the malignant tumor cells.

Immune checkpoint therapy such as checkpoint inhibitors include, but are not limited to programmed death-1 (PD-1) inhibitors, programmed death ligand-1 (PD-L1) inhibitors, programmed death ligand-2 (PD-L2) inhibitors, lymphocyte-activation gene 3 (LAG3) inhibitors, T-cell immunoglobulin and mucin-domain containing protein 3 (TIM-3) inhibitors, T cell immunoreceptor with Ig and ITIM domains (TIGIT) inhibitors, B- and T-lymphocyte attenuator (BTLA) inhibitors, V-domain Ig suppressor of T-cell activation (VISTA) inhibitors, cytotoxic T-lymphocyte-associated protein 4 (CTLA4) inhibitors, Indoleamine 2,3-dioxygenase (IDO) inhibitors, killer immunoglobulin-like receptors (KIR) inhibitors, KIR2L3 inhibitors, KIR3DL2 inhibitors and carcinoembryonic antigen-related cell adhesion molecule 1 (CEACAM-1) inhibitors. In particular, checkpoint inhibitors include antibodies anti-PD1, anti-PD-L1, anti-CTLA-4, anti-TIM-3, anti-LAG3. Immune checkpoint therapy also include co-stimulatory antibodies delivering positive signals through immune-regulatory receptors including but not limited to ICOS, CD137, CD27, OX-40 and GITR.

Example of anti-PD1 antibodies include, but are not limited to, nivolumab, cemiplimab (REGN2810 or REGN-2810), tislelizumab (BGB-A317), tislelizumab, spartalizumab (PDR001 or PDR-001), ABBV-181, JNJ-63723283, BI 754091, MAG012, TSR-042, AGEN2034, pidilizumab, nivolumab (ONO-4538, BMS-936558, MDX1106, GTPL7335 or Opdivo), pembrolizumab (MK-3475, MK03475, lambrolizumab, SCH-900475 or Keytruda) and antibodies described in International patent applications WO2004004771, WO2004056875, WO2006121168, WO2008156712, WO2009014708, WO2009114335, WO2013043569 and WO2014047350. Example of anti-PD-L1 antibodies include, but are not limited to, LY3300054, atezolizumab, durvalumab and avelumab. Example of anti-CTLA-4 antibodies include, but are not limited to, ipilimumab (see, e.g., US patents U.S. Pat. Nos. 6,984,720 and 8,017,114), tremelimumab (see, e.g., U.S. Pat. Nos. 7,109,003 and 8,143,379), single chain anti-CTLA4 antibodies (see, e.g., International patent applications WO1997020574 and WO2007123737) and antibodies described in US patent U.S. Pat. No. 8,491,895. Example of anti-VISTA antibodies are described in US patent application US20130177557. Example of inhibitors of the LAG3 receptor are described in US patent U.S. Pat. No. 5,773,578. Example of KIR inhibitor is IPH4102 targeting KIR3DL2.

In some embodiments, the compound and/or pharmaceutical composition of the invention may be used in combination with targeted therapy. As used herein, the term “targeted therapy” refers to targeted therapy agents, drugs designed to interfere with specific molecules necessary for tumor growth and progression. For example, targeted therapy agents such as therapeutic monoclonal antibodies target specific antigens found on the cell surface, such as transmembrane receptors or extracellular growth factors. Small molecules can penetrate the cell membrane to interact with targets inside a cell. Small molecules are usually designed to interfere with the enzymatic activity of the target protein such as for example proteasome inhibitor, tyrosine kinase or cyclin-dependent kinase inhibitor, histone deacetylase inhibitor. Targeted therapy may also use cytokines. Examples of such targeted therapy include with no limitations: Ado-trastuzumab emtansine (HER2), Afatinib (EGFR (HER1/ERBB1), HER2), Aldesleukin (Proleukin), alectinib (ALK), Alemtuzumab (CD52), axitinib (kit, PDGFRbeta, VEGFR1/2/3), Belimumab (BAFF), Belinostat (HDAC), Bevacizumab (VEGF ligand), Blinatumomab (CD19/CD3), bortezomib (proteasome), Brentuximab vedotin (CD30), bosutinib (ABL), brigatinib (ALK), cabozantinib (FLT3, KIT, MET, RET, VEGFR2), Canakinumab (IL-1 beta), carfilzomib (proteasome), ceritinib (ALK), Cetuximab (EGFR), cofimetinib (MEK), Crizotinib (ALK, MET, ROS1), Dabrafenib (BRAF), Daratumumab (CD38), Dasatinib (ABL), Denosumab (RANKL), Dinutuximab (B4GALNT1 (GD2)), Elotuzumab (SLAMF7), Enasidenib (IDH2), Erlotinib (EGFR), Everolimus (mTOR), Gefitinib (EGFR), Ibritumomab tiuxetan (CD20), Sonidegib (Smoothened), Sipuleucel-T, Siltuximab (IL-6), Sorafenib (VEGFR, PDGFR, KIT, RAF), (Tocilizumab (IL-6R), Temsirolimus (mTOR), Tofacitinib (JAK3), Trametinib (MEK), Tositumomab (CD20), Trastuzumab (HER2), Vandetanib (EGFR), Vemurafenib (BRAF), Venetoclax (BCL2), Vismodegib (PTCH, Smoothened), Vorinostat (HDAC), Ziv-aflibercept (PIGF, VEGFA/B), Olaparib (PARP inhibitor).

In some embodiments, the compound and/or pharmaceutical composition of the invention may be used in combination with chemotherapy. As used herein, the term “antitumor chemotherapy” or “chemotherapy” has its general meaning in the art and refers to a cancer therapeutic treatment using chemical or biochemical substances, in particular using one or several antineoplastic agents or chemotherapeutic agents. Chemotherapeutic agents include, but are not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; methylhydrazine derivatives including N-methylhydrazine (MIH) and procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylomithine (DMFO); retinoids such as retinoic acid; capecitabine; anthracyclines, nitrosoureas, antimetabolites, epipodophylotoxins, enzymes such as L-asparaginase; anthracenediones; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide; and pharmaceutically acceptable salts, acids or derivatives of any of the above.

In some embodiments, the compound and/or pharmaceutical composition of the invention is administered to the subject in combination with radiotherapy. Suitable examples of radiation therapies include, but are not limited to external beam radiotherapy (such as superficial X-rays therapy, orthovoltage X-rays therapy, megavoltage X-rays therapy, radiosurgery, stereotactic radiation therapy, Fractionated stereotactic radiation therapy, cobalt therapy, electron therapy, fast neutron therapy, neutron-capture therapy, proton therapy, intensity modulated radiation therapy (IMRT), 3-dimensional conformal radiation therapy (3D-CRT) and the like); brachytherapy; unsealed source radiotherapy; tomotherapy; and the like. Gamma rays are another form of photons used in radiotherapy. Gamma rays are produced spontaneously as certain elements (such as radium, uranium, and cobalt 60) release radiation as they decompose, or decay. In some embodiments, radiotherapy may be proton radiotherapy or proton minibeam radiation therapy. Proton radiotherapy is an ultra-precise form of radiotherapy that uses proton beams (Prezado Y, Jouvion G, Guardiola C, Gonzalez W, Juchaux M, Bergs J, Nauraye C, Labiod D, De Marzi L, Pouzoulet F, Patriarca A, Dendale R. Tumor Control in RG2 Glioma-Bearing Rats: A Comparison Between Proton Minibeam Therapy and Standard Proton Therapy. Int J Radiat Oncol Biol Phys. 2019 Jun. 1; 104 (2): 266-271. doi: 10.1016/j.ijrobp.2019.01.080; Prezado Y, Jouvion G, Patriarca A, Nauraye C, Guardiola C, Juchaux M, Lamirault C, Labiod D, Jourdain L, Sebrie C, Dendale R, Gonzalez W, Pouzoulet F. Proton minibeam radiation therapy widens the therapeutic index for high-grade gliomas. Sci Rep. 2018 Nov. 7; 8 (1): 16479. doi: 10.1038/s41598-018-34796-8). Radiotherapy may also be FLASH radiotherapy (FLASH-RT) or FLASH proton irradiation. FLASH radiotherapy involves the ultra-fast delivery of radiation treatment at dose rates several orders of magnitude greater than those currently in routine clinical practice (ultra-high dose rate) (Favaudon V, Fouillade C, Vozenin M C. The radiotherapy FLASH to save healthy tissues. Med Sci (Paris) 2015; 31:121-123. DOI: 10.1051/medsci/20153102002); Patriarca A., Fouillade C. M., Martin F., Pouzoulet F., Nauraye C., et al. Experimental set-up for FLASH proton irradiation of small animals using a clinical system. Int J Radiat Oncol Biol Phys, 102 (2018), pp. 619-626. doi: 10.1016/j.ijrobp.2018.06.403. Epub 2018 Jul. 11).

### Pharmaceutical Composition for Use in Treating DDLPS

The compounds of the invention may be used or prepared in a pharmaceutical composition.

In a second aspect, the invention relates to a pharmaceutical composition comprising at least one TGFβ inhibitor and at least one pharmaceutical acceptable carrier for use in the treatment of cancer in a subject of need thereof.

In some embodiments, the invention relates to a pharmaceutical composition comprising a compound of the invention and a pharmaceutical acceptable carrier for use in the treatment of a dedifferentiated liposarcoma (DDLPS) in a subject in need thereof.

In some embodiments, the invention relates to a pharmaceutical composition comprising the compound of the invention and a pharmaceutical acceptable carrier for use in the treatment of an advanced dedifferentiated liposarcoma (DDLPS) in a subject in need thereof.

Typically, the compound of the invention may be combined with pharmaceutically acceptable excipients, and optionally sustained-release matrices, such as biodegradable polymers, to form therapeutic compositions.

Typically, the compounds according to the invention as described above are administered to the subject in a therapeutically effective amount.

By a “therapeutically effective amount” of the compound of the present invention as above described is meant a sufficient amount of the compound at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidential with the specific compound employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the compound of the present invention for the symptomatic adjustment of the dosage to the patient to be treated. A medicament typically contains from about 0.01 mg to about 500 mg of the compound of the present invention, preferably from 1 mg to about 100 mg of the compound of the present invention. An effective amount of the drug is ordinarily supplied at a dosage level from 0.0002 mg/kg to about 20 mg/kg of body weight per day, especially from about 0.001 mg/kg to 7 mg/kg of body weight per day.

In a particular embodiment, the compound according to the invention may be used in a concentration between 0.01 μM and 20 μM, particularly, the compound of the invention may be used in a concentration of 0.01, 0.05, 0.1, 0.5, 1.0, 2.5, 5.0, 10.0, 15.0, 20.0 μM.

According to the invention, the compound of the present invention is administered to the subject in the form of a pharmaceutical composition. Typically, the compound of the present invention may be combined with pharmaceutically acceptable excipients, and optionally sustained-release matrices, such as biodegradable polymers, to form therapeutic compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Typically, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The compound of the present invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized agents of the present inventions into a sterile vehicle which contains the basic dispersion medium and the required other ingredients from those enumerated above. In the case of sterile powders for the preparation of sterile injectable solutions, the typical methods of preparation are vacuum-drying and freeze-drying techniques which yield a powder of the compound of the present invention plus any additional desired ingredient from a previously sterile-filtered solution thereof. The preparation of more, or highly concentrated solutions for direct injection is also contemplated, where the use of DMSO as solvent is envisioned to result in extremely rapid penetration, delivering high concentrations of the active agents to a small tumor area. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

Pharmaceutical compositions of the invention may include any further compound which is used in the treatment of cancer such as described above.

In some embodiments, the pharmaceutical compositions of the invention may include any further compound which is used in the treatment of a dedifferentiated liposarcoma (DDLPS).

In one embodiment, said additional active compounds may be contained in the same composition or administrated separately.

In another embodiment, the pharmaceutical composition of the invention relates to combined preparation for simultaneous, separate or sequential use in the treatment of cancer in a subject in need thereof.

In some embodiments, the pharmaceutical composition of the invention relates to a combined preparation for simultaneous, separate or sequential use in the treatment of dedifferentiated liposarcoma (DDLPS) in a subject in need thereof.

The invention also provides kits comprising the compound of the invention. Kits containing the compound of the invention find use in therapeutic methods.

The invention will be further illustrated by the following examples. However, these examples should not be interpreted in any way as limiting the scope of the present invention.

### Example

Single cell transcriptomic landscape of DDLPS. Unbiaised clustering using Seurat identified 42 main clusters based on uniform manifold approximation and projection (UMAP). Gene expression profiles and expression of canonical markers enabled to gather these clusters into 10 main cellular subfamilies, including tumoral cells, myeloid cells, lymphoid cells, endothelial cells, red blood cells, pericytes, mast cells, mesothelial cells, adipocytes, and cycling cells (Data not shown).

Through RNA-sequencing of 102,753 individual cells from 11 primary DDLPS lesions, major cell clusters were identified based on unsupervised clustering of gene expression profiles and canonical markers. They include 31 tumor microenvironment clusters and 11 tumor cell clusters.

Single cell transcriptomic landscape of tumor cells coming from the WD compartment. Unbiaised clustering using Seurat identified 11 main clusters, including two clusters characterized by a stemness signature. This stemness signature corresponds to signatures of early adipocytic progenitors previously describes in Merrick et al and Hemont et al. These cells express a specific set of markers genes including CD44, CD55, DPP4 and FBN1. CD44 staining by immunohistochemistry confirms the presence of spindle, undifferentiated tumor cells interspersed between tumor adipocytes in the WD compartment of DDLPS corresponding to these presumed adipocytic progenitors (Data not shown).

A cluster of tumor cells from the WD compartment is characterized by signatures of early adipocytic progenitors, previously identified as TGFβ-dependent, DPP4-positive stromal progenitors.

Genomic characterization of DDLPS tumor cell populations. Inference of copy number variations in a patients' tumor using InferCNV shows that tumor progenitors harbor ancestral genomic alterations including 12q amplification and 1q amplification that are the only alterations common in both WD and DD compartments. Thus, this shows that WD and DD components are not related but derive from the same precursor.

The inventors show that these cells harbor specifically the truncal genomic alterations of the cancer, with further subclonal mutations identifiable in both WD and DD compartments of DDLPS.

DDLPS tumor cells keep the multipotent properties of their progenitors. Ex vivo differentiation of tumor cells from 2 DDLPS PDX models towards the adipocytic and osteogenic lineage shows that DD cells keep multipotent properties similar to their progenitors. Adipocytic differentiation is confirmed with the detection of green vacuoles (Bodipy staining) and osteogenic differentiation by red staining after Alizarin red treatment (Data not shown).

Furthermore, these cells have multipotent properties and their differentiation towards the adipocytic lineage is inhibited by TGFβ. DD tumor cells keep functional properties of their progenitors, including multipotency.

DDLPS tumor cells are vulnerable to TGFβ inhibition.

It is shown for the time that treatment of DD tumor cells with TGFβ impairs adipocytic differentiation of DD cells, whereas TGFβ inhibitors restore the adipocytic phenotype of these cells in vitro.

Ex vivo adipocytic differentiation of DD tumor cells from 2 DDLPS PDX models is prevented in the presence of TGFβ both at the transcriptomic and phenotypic level, whereas treatment with TGFβ inhibitors galunisertib or SB431542 restores adipocytic differentiation.

The inventors also investigated the role of TGFβ in adipocytic differentiation in DDLPS, particularly advanced DDLPS using several TGFβ inhibitors, of a different mode of action and of different nature, such as compounds selected from the group consisting of TGFβ receptor R1/R2 inhibitors LY2109761 and LY364947; monoclonal anti-TGFβ antibodies BX-BE0057 and fresolimumab; TGFβ ligand trap such as luspatercept (a fusion protein that traps TGFβ ligands preventing them from binding to Type II TGFβ receptors) and bintrafusp alfa (a Bifunctional Fusion Protein Targeting TGF-β and PD-L1) and TQB2858 (an anti-TGF-β/PD-L1 antibody). Tested compounds are found at least as effective than galunisertib or SB431542 in inhibiting ex vivo TGFβ pathway in DDLPS PDX Sil10AS, also they are found to restore adipocytic differentiation phenotype of DD cells.

7 nude mice engrafted with the DDLPS PDX Sil10AS are treated with TGFβ inhibitor galunisertib. The tumor growth rate is compared to a control group of 7 nude mice engrafted with the same PDX model and treated with a vehicle. At the end of the experiment, tumor differentiation are assessed at the microscopic and transcriptomic level.

The treatment of DDLPS patient-derived xenografts (PDX) with inhibitors of TGFβ reduces tumor growth and restores adipocytic differentiation of DD cells.

### REFERENCES

Throughout this application, various references describe the state of the art to which this invention pertains. The disclosures of these references are hereby incorporated by reference into the present disclosure.
Watson S, Gruel N, Le Loarer F. New developments in the pathology and molecular biology of retroperitoneal sarcomas. Eur J Surg Oncol. 2022 Feb. 5: S0748-7983 (22) 00078-6.
Gruel N, El Zein S, Tzanis D, Nicolas N, Maraval A, Fieffe C, Bonvalot S, Caly M, Fuhrmann L, Ait Rais K, Jovelin S, Bonnet C, Pierron G, Watson S. MDM4 amplification in atypical lipomatous tumors/well-differentiated liposarcoma: Private event or alternative oncogenic mechanism? Genes Chromosomes Cancer. 2023 Feb. 6.
Huang C Y, Chung C L, Hu T H, Chen J J, Liu P F, Chen C L. Recent progress in TGF-β inhibitors for cancer therapy. Biomed Pharmacother. 2021 February; 134:111046.
Xie L, Liang X, Xu J, Sun X, Liu K, Sun K, Li Y, Tang X, Li X, Zhan X, Niu X, Guo W. Exploratory study of an anti-PD-L1/TGF-β antibody, TQB2858, in patients with refractory or recurrent osteosarcoma and alveolar soft part sarcoma: a report from Chinese sarcoma study group (TQB2858-Ib-02). BMC Cancer. 2023 Sep. 15; 23 (1): 868.

1. A TGFβ inhibitor for use in the treatment of a dedifferentiated liposarcoma (DDLPS) in a subject in need thereof.

2. The TGFβ inhibitor for use according to claim 1, said TGFβ inhibitor being selected from: antisense oligonucleotides preventing TGF-β synthesis, ligand traps and neutralizing antibodies preventing a TGF-β ligand from binding to receptors; small molecule TGF-β inhibitors that inhibit receptor kinase activity and/or prevent TGFβ receptor mediated signal transduction.

3. The TGFβ inhibitor for use according to claim 1, said TGFβ inhibitor being selected from galunisertib, SB431542, 2G7, 1D11, LY2109761, IN-1130, LY364947, PF-03446962, Ki 26894, SD208, SB505124, LY550410, AP12009, AP1104, AP15012, ISTH0036, fresolimumab, LY2382770, BX-BE0057, luspatercept, bintrafusp alfa, TQB2858, CAT-192, P144, belagenpumatucel-L and/or gemogenovatucel-T, or a combination thereof.

4. The TGFβ inhibitor for use according to claim 1, wherein the dedifferentiated liposarcoma is an advanced dedifferentiated liposarcoma.

5. A pharmaceutical composition comprising at least one TGFβ inhibitor and at least one pharmaceutical acceptable carrier for use in the treatment of a dedifferentiated liposarcoma in a subject in need thereof.

6. The pharmaceutical composition for use according to claim 5, wherein the at least one TGFβ inhibitor is selected from galunisertib, SB431542, 2G7, 1D11, LY2109761, IN-1130, LY364947, PF-03446962, Ki 26894, SD208, SB505124, LY550410, AP12009, AP1104, AP15012, ISTH0036, fresolimumab, LY2382770, BX-BE0057, luspatercept, bintrafusp alfa, TQB2858, CAT-192, P144, belagenpumatucel-L or gemogenovatucel-T, or a combination thereof.

7. The pharmaceutical composition for use according to claim 5 wherein the dedifferentiated liposarcoma is an advanced dedifferentiated liposarcoma.

8. A method of treating a dedifferentiated liposarcoma in a subject in need thereof, comprising administering to the subject a therapeutically effective amount of at least one TGFβ inhibitor.

9. The method of treating a dedifferentiated liposarcoma in a subject in need thereof according to claim 8 wherein the at least one TGFβ inhibitor selected from galunisertib, 2G7, 1D11, LY2109761, IN-1130, LY364947, PF-03446962, SB431542, Ki 26894, SD208, SB505124, LY550410, AP12009, AP1104, AP15012, ISTH0036, fresolimumab, LY2382770, BX-BE0057, luspatercept, bintrafusp alfa, TQB2858, CAT-192, P144, belagenpumatucel-L or gemogenovatucel-T, or a combination thereof.
